(19) United States
(12) Patent Application Publication
(10) Pub. No.: US 2023/0270898 A1
(43) Pub. Date: Aug. 31, 2023

(54) ULTRAVIOLET LIGHT IRRADIATION SYSTEM AND DECONTAMINATION METHOD

(71) Applicant: NIPPON TELEGRAPH AND TELEPHONE CORPORATION, Tokyo (JP)

(72) Inventors: Takashi MATSUI, Musashino-shi, Tokyo (JP); Kazuhide NAKAJIMA, Musashino-shi, Tokyo (JP); Nobutomo HANZAWA, Musashino-shi, Tokyo (JP); Yuto SAGAE, Musashino-shi, Tokyo (JP); Chisato FUKAI, Musashino-shi, Toyo (JP); Ayako IWAKI, Musashino-shi, Tokyo (JP); Tomohiro TANIGUCHI, Musashino-shi, Tokyo (JP); Kazutaka HARA, Musashino-shi, Tokyo (JP); Atsuko KAWAKITA, Musashino-shi, Tokyo (JP)

(73) Assignee: NIPPON TELEGRAPH AND TELEPHONE CORPORATION, Tokyo (JP)

(21) Appl. No.: 18/018,038

(22) PCT Filed: Oct. 23, 2020
(86) PCT No.: PCT/JP2020/039818

§ 371 Date: Jan. 25, 2023

(30) Foreign Application Priority Data
Jul. 30, 2020 (WO) PCT/JP2020/029268

(51) Int. Cl. A61L 2/10; A61L 9/20

(52) U.S. Cl. CPC A61L 2/10; A61L 9/20; A61L 2202/11; A61L 2202/15

(57) ABSTRACT

An object of the present invention is to provide an ultraviolet light irradiation system and a decontamination method that can perform decontamination economically without any input from a user.

The ultraviolet light irradiation system forms a linear or planar ultraviolet light irradiation space by spatially bundling a plurality of ultraviolet light beams with high energy density or moving the ultraviolet light with high energy density at high speed. The ultraviolet light irradiation system can decontaminate a human body and clothing simply by passing through the space. Further, since the ultraviolet light irradiation system performs decontamination in the space, bacteria and viruses emitted from a carrier are not passed through the space.

### TECHNICAL FIELD

The present disclosure relates to an ultraviolet light irradiation system and a decontamination method for performing sterilization and virus inactivation using ultraviolet light.

### BACKGROUND ART

For the purpose of preventing infectious diseases, there is an increasing demand for systems with ultraviolet light for sterilization and virus inactivation using ultraviolet light. In the present embodiment, ‘decontamination is assumed to include sterilization and virus inactivation.

There are three major categories of products in decontamination systems.

### (1) Noble Sterilization Robot

A mobile sterilization robot is an autonomous mobile robot for radiating ultraviolet light, (for example, refer to Non Patent Literature 1). The mobile sterilization robot radiates ultraviolet light while moving in a room in a building such as a hospital room, thereby automatically performing decontamination in a wide range without manual operation.

### (2) Stationary Air Purifier

A stationary air purifier is a device that is installed on a ceiling or in a predetermined place in a room and decontaminates while circulating the air in the room (for example, refer to Non Patent Literature 2). Since a stationary air purifier does not emit ultraviolet light to the outside and has no influence on the human body, it is capable of highly safe decontamination.

### (3) Portable Sterilization Device

A portable sterilization device is a portable type device mounted with an ultraviolet light source such as a fluorescent lamp, a mercury lamp, or an LED (for example, refer to Non Patent Literature 3) A user brings a portable sterilization device to an area that the user wants to decontaminate, and irradiates the area with ultraviolet light. In this way, a portable sterilization device can be used in various locations.

### CITATION LIST

### Non Patent Literature

[NPL 1] Website of Kantum Ushikata Co., LTD. (https://www.kantum.co.jp/product/sakkin_robot/sakkinn_robot/UVD_r obot), search on Jul. 3, 2020
[NPL 2] Website of IWASAKI ELECTRIC CO., LTD. (https://www.iwasaki.co.jp/optics/sterilization/air/air03.html), search on Jul. 3, 2020
[NPL 3] Website of Funakoshi Co., Ltd. (https://www.funakoshi.co.jp/contents/68182), search on Jul. 3, 2020

### SUMMARY OF INVENTION

### Technical Problem

In consideration of a life style, it is preferable that decontamination can be performed without being attached to a human body or clothing or without being aware of bacteria and viruses that are unintentionally released from carriers. However, the purpose of the techniques disclosed so far is to specify an object and a range, to perform decontamination limited thereto, and it is difficult to create a preferable state in which the decontamination can be performed without any input from a user.

The prior art further has the following difficulties.
(1) Since the mobile sterilization robot radiates high-power ultraviolet light, the device is large and expensive. Therefore, it is difficult to economically realize the mobile type sterilization robot.
(2) Since the stationary air purifier uses a method of sterilizing the circulated indoor air, there is a problem that it is difficult to decontaminate clothes and the like and to immediately decontaminate bacteria and viruses emitted from carriers.
(3) The portable sterilization device has a problem that the irradiated ultraviolet rays are relatively weak and it is difficult to use it to decontaminate in a short time. Even if a mercury lamp or a fluorescent lamp with high output is used, these lamps are generally large-sized and short-lived, and since light is diffused in proportion to the square of the distance to reduce the power, it is difficult to apply these lamps to a portable sterilization device.

Although implementation of sterilization systems and methods for solving these problems are expected, no specific means has come to light. Therefore, in order to solve the above problems, an object of the present invention is to provide an ultraviolet light irradiation system and a decontamination method that are economical, and that can perform decontamination without any input from a user.

### Solution to Problem

In order to achieve the above object, the ultraviolet light irradiation system according to the present invention forms a curtain of ultraviolet light in a space.

Specifically, a first ultraviolet light irradiation system according to the present invention is an ultraviolet light irradiation system including ultraviolet light irradiation units for guiding ultraviolet light in a collimated state in a desired space, in which the ultraviolet light irradiation units are arranged on a straight line or a plane at arbitrary intervals, and each of the ultraviolet light irradiation units includes an ultraviolet light source unit that emits the ultraviolet light and a condensing component that causes the ultraviolet light incident from the ultraviolet light source unit directly or via an optical fiber to become ultraviolet light in the collimated state.

In addition, a second ultraviolet light irradiation system according to the present invention is an ultraviolet light irradiation system including ultraviolet light irradiation units that guide ultraviolet light in a collimated state into a desired space, in which

the ultraviolet light irradiation unit includes
one ultraviolet light source unit,
a plurality of condensing components arranged on a straight line or a plane at arbitrary intervals and emitting the supplied ultraviolet light as ultraviolet light in the collimated state,
a branch switch unit that branches the ultraviolet light output from the ultraviolet light source unit and supplies the branched ultraviolet light to the respective condensing components, or
supplies the ultraviolet light output from the ultraviolet light source unit to the respective condensing components in order, and an optical fiber for supplying the ultraviolet light output from the ultraviolet light source unit to the condensing component.

Further, a third ultraviolet light irradiation system according to the present invention is an ultraviolet light irradiation system including ultraviolet light irradiation units that guide ultraviolet light in a collimated state into a desired space, in which

the ultraviolet light irradiation unit includes
one ultraviolet light source unit,
one condensing component that emits the ultraviolet light output from the ultraviolet light source unit as ultraviolet light in the collimated state,
an optical fiber for supplying the ultraviolet light output from the ultraviolet light source unit to the condensing component, and
a drive control unit that causes the condensing component to scan on a straight line or a plane.

In addition, a decontamination method according to the present invention includes guiding ultraviolet light in a collimated state into a desired space, and sterilizing a human body or an object passing through the desired space or inactivating a virus or blocking bacteria or a virus in the desired space.

The ultraviolet light irradiation system forms a linear or planar ultraviolet light irradiation space (a curtain of ultraviolet light) by spatially bundling a plurality of ultraviolet light beams with high energy density or moving the ultraviolet light with high energy density at a high speed. With the ultraviolet light irradiation system, a human body and clothing can be decontaminated simply by passing through the space. Further, since the ultraviolet light irradiation system performs decontamination in the space, bacteria and viruses emitted from a carrier are not passed through the space.

That is, the ultraviolet light irradiation system can perform decontamination only when something is passed through the irradiation space of the ultraviolet light. In addition, the ultraviolet light irradiation system can divide an area in an ultraviolet light irradiation space, and prevent propagation of bacteria and viruses across the space. Thus, the ultraviolet light irradiation system is simple and can prevent infection with bacteria and viruses without any input from user. Accordingly, the present invention can provide an ultraviolet light irradiation system and a decontamination method that can perform decontamination economically without any input from a user.

The ultraviolet light irradiation system according to the present invention further includes

a sensor for sensing an object to be irradiated in the desired space, and
an irradiation control unit that controls output or non-out put of ultraviolet light from the ultraviolet light source unit based on a signal of the sensor.

Safety can be improved and the service life of the equipment can be prolonged.

The ultraviolet light irradiation system according to the present invention further includes a display unit configured to display an output state of the ultraviolet light of the ultraviolet light source unit. Thus, it can be clearly indicated that it is in operation for improved safety.

The optical fiber of the ultraviolet light irradiation system according to the present invention includes any one of a solid core optical fiber, a hole assist optical fiber, a hole structure optical fiber, a hollow core optical fiber, a coupled core type optical fiber, a solid core type multi-core optical fiber, a hole assist type multi-core optical fiber, a hole structure type multi-core optical fiber, a hollow core type multi-core optical fiber, and a coupled core type multi-core optical fiber. The optical fiber can increase a transmission light intensity of the ultraviolet light and reduce a leakage loss at a bending portion or the like.

The ultraviolet light in the collimated state of the ultraviolet light irradiation system according to the present invention is collimated by a collimator lens, in which the collimator lens is an optical fiber whose tip is processed into a spherical shape or an optical fiber having a graded refractive index distribution at the tip.

The inventions can be combined with each other where possible.

### Advantageous Effects of Invention

The present invention can provide an ultraviolet light irradiation system and a decontamination method that are economical and that can perform decontamination without any input from a user.

### BRIEF DESCRIPTION OF DRAWINGS

FIG. 1 is a diagram illustrating an ultraviolet light irradiation system according to the present invention.

FIG. 2 is a diagram illustrating the ultraviolet light irradiation system according to the present invention.

FIG. 3 is a diagram illustrating the ultraviolet light irradiation system according to the present invention.

FIG. 4 is a diagram illustrating an optical fiber of the ultraviolet light irradiation system according to the present invention.

### DESCRIPTION OF EMBODIMENTS

Embodiments of the present invention will be described with reference to the accompanying drawings. The embodiments described below/are examples of the present invention, and the present invention is not limited to the following embodiments. Note that, in the present specification and the drawings, the components having the same reference numerals indicate the sane components.

### Embodiment 1

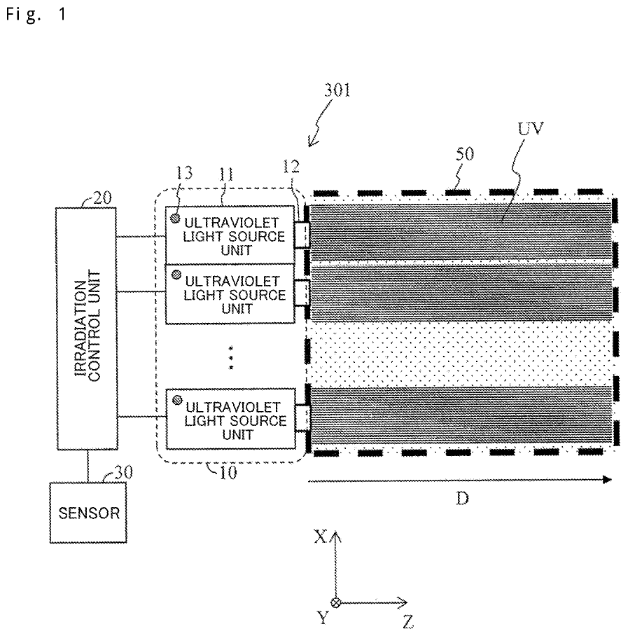

FIG. 1 is a diagram illustrating an ultraviolet light irradiation system 301 of the present embodiment. The ultraviolet light irradiation system 301 includes an ultraviolet light irradiation unit 10 for guiding ultraviolet light UV in a collimated state in a desired space 50, in which the ultraviolet light irradiation unit 10 includes a plurality of ultraviolet light source units 11 arranged on a straight line or a plane at arbitrary intervals and emitting the ultraviolet light UV in the collimated state.

Each of the ultraviolet light source units 11 emits light waves in a deep ultraviolet wavelength region having a wavelength of 200 to 300 nm. Specifically, light waves with a wavelength of 222 nm are preferable because they are known to have a sufficiently small influence on the human body. The ultraviolet light source unit 11 may include a light source having a wavelength longer than that of ultraviolet light and a harmonic generator. For example, the ultraviolet light source unit 11 may include a high-out put light source of a 1064 nm band and a fourth or fifth harmonic generator.

The light waves emitted from the ultraviolet light source unit 11 are converted into the ultraviolet light UV in the collimated state which is small in spread and spatially propagated with high energy density by passing through a condensing component 12. The condensing component 12 is a component such as a collimator lens, a GRIN lens, or a concave mirror for condensing spherical waves into linear light. An ultraviolet light source unit 11 and the condensing component 12 may be connected through an optical fiber. In this case, it is not necessary to arrange the ultraviolet light source unit 11 in a room to be decontaminated. By connecting the ultraviolet light source unit 11 and the condensing component 12 by an optical fiber, the degree of freedom of design of the ultraviolet light irradiation system can be increased.

The ultraviolet light UV in the collimated state attenuates according to the propagating distance. Therefore, the ultraviolet light source unit 11 outputs light waves having an intensity for allowing the collimated ultraviolet light UV to reach a desired depth D in the space 50. That is, the depth D of the space 50 can be adjusted by the light output power of the ultraviolet light source unit 11.

When the plurality of ultraviolet light source units 11 are arranged in a line in an X direction (the ultraviolet light source units 11 are arranged on a straight line),

in a Y-direction, a width of one ultraviolet light UV is the space 50,
in the X-direction, a width corresponds to the number of ultraviolet light UV arranged is the space 50, and
in a Z-direction, a distance that the ultraviolet light UV can reach (distance to maintain light intensity that can be decontaminated) is the space 50.

In the X-direction, a gap is provided between the adjacent ultraviolet light UV, or a part of the adjacent ultraviolet light UV is disposed to be overlapped in addition to adjusting the number of the ultraviolet light source units 11, thereby adjusting the length of the space 50 in the X-direction.

Thus, by arranging the plurality of ultraviolet light source units 11 in a line in the X-direction, a curtain-like space 50 of the ultraviolet light can be formed.

In addition, when the ultraviolet light source units 11 are also arranged in the Y-direction (when the ultraviolet light source units 11 is arranged on a plane), the width in the Y-direction of the space 50 can be widened to the width equivalent to several of the arranged ultraviolet light UV. That is, the thickness of the curtain of the ultraviolet light can be increased.

Since the space 50 is irradiated with the ultraviolet light UV, decontamination can be performed. That is, the ultraviolet light irradiation system 301 is arranged at an arbitrary place to form the space 50 which is a curtain of the ultraviolet light, and the human body or the object can be decontaminated simply by passing through the space 50. In addition, since bacteria and viruses cannot come and go through the space 50 which is a curtain of ultraviolet light, the ultraviolet light irradiation system 301 is arranged in a room larger than the space 50, and the room can be divided with respect to bacteria and viruses in the space 50. Specifically, one room can be divided with respect to a decontamination area and a contaminated area.

The ultraviolet light irradiation system 301 may further include a sensor 30 for sensing an object to be irradiated in the desired space 50 and an irradiation control unit 20 for controlling output or non-out put of ultraviolet light from the ultraviolet light source unit 11 based on a signal of the sensor 30.

By providing the irradiation control unit 20, irradiation or non-irradiation of the ultraviolet light UV at an arbitrary timing can be performed, which is preferable since it is possible to improve safety and prolong the service life of the ultraviolet light source unit 11.

In addition, in the case of using a wavelength such as a UV-C region (a wavelength of 100 to 280 nm) as the ultraviolet light UV that may affect the human body, the irradiation control unit 20 may perform the following control. When the sensor 30 senses a decontamination object as an object, the irradiation control unit 20 turns on the ultraviolet light source unit 11, and when a person is sensed or there is no decontamination object, the irradiation control unit 20 turns off the ultraviolet light source unit 11.

The ultraviolet light irradiation system 301 further includes a display unit 13 for displaying an output state of the ultraviolet light of the ultraviolet light source unit 11. The display unit 13 clearly indicates that the ultraviolet light source unit 11 is outputting the ultraviolet light. For example, the display unit 13 is a visible light source, and can be clearly visually indicated by emitting visible light in conjunction with the ultraviolet light source unit 11.

In the present embodiment, the embodiment in which the ultraviolet light source units 11 are arranged on a straight line or a plane and the ultraviolet light UV is irradiated in one direction (Z-direction) has been described. However, the ultraviolet light source unit 11 may be arranged so that the ultraviolet light UV can be irradiated from a plurality of directions (not only from the Z-direction but also from the Y-direction).

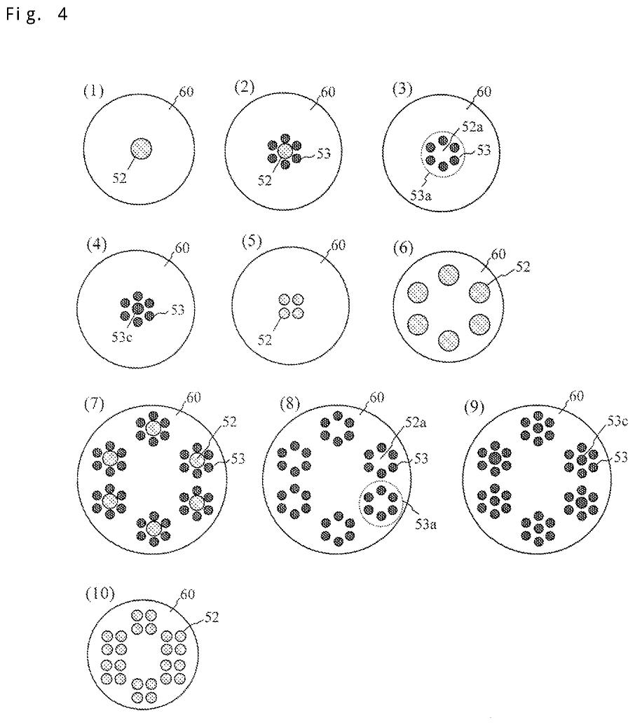

Although the ultraviolet light from the ultraviolet light source unit 11 is directly coupled to the condensing component 12 in the present embodiment, the ultraviolet light source unit 11 and the condensing component 12 may be connected to each other through an optical fiber. As the optical fiber, an optical fiber having a cross section as illustrated in FIG. 4 can be used. In addition to the solid type optical fiber using a general additive such as that illustrated in (1) of FIG. 4, optical fibers having a hole structure described in (2) to (4) of FIG. 4, multi-core optical fibers having a plurality of core regions described in (5) and (6) of FIG. 4, or optical fibers having a structure obtained by combining them ((7) to (10) of FIG. 4) may be used.

FIG. 4 is a diagram illustrating the optical fiber.

### (1) Solid Core Optical Fiber

The optical fiber has one solid core 52 with a refractive index higher than that of a clad 60 in the clad 60. The ‘solid means ‘not a cavity. The solid core can also be realized by forming an annular low refractive index region in the clad.

### (2) Hole Assist Optical Fiber

The optical fiber has the solid core 52 and a plurality of holes 53 disposed on the outer periphery of the solid core 52 in the clad 60. The medium of the hole 53 is air, and the refractive index of the air is sufficiently smaller than that of quartz glass. Therefore, the hole assist optical fiber has a function of returning light leaked from the core 52 by bending or the like to the core 52 again, and has a characteristic of small bending loss.

### (3) Hole Structure Optical Fiber

The optical fiber has a hole group 53a of a plurality of the holes 53 in the clad 60, and has a refractive index effectively lower than that of a host material (glass or the like). This structure is called a photonic crystal fiber. This structure can have a structure in which a high refractive index core having a varied refractive index does not exist, and light can be confined by making a region 52a surrounded by the holes 53 as an effective core region. As compared with an optical fiber having a solid core, a photonic crystal fiber can reduce the influence of absorption and scattering loss of the core by an additive, and can realize optical characteristics which cannot be realized by the solid optical fiber such as reduction of bending loss and control of nonlinear effect.

### (4) Hollow Core Optical Fiber

The core region of the optical fiber is formed with air. The light can be confined in the core region by taking a photonic band gap structure by a plurality of holes or an anti-resonant structure by a glass thin wire in the clad region. This optical fiber has a small nonlinear effect and can supply a high output or high energy laser.

### (5) Coupled Core Type Optical Fiber

In this optical fiber, a plurality of the solid cores 52 with a high refractive index are disposed in close proximity to each other in the clad 60. The optical fiber guides light by light wave coupling between the solid cores 52. Since the coupled core type optical fiber can disperse and send light by the number of cores, it is possible to increase the power and sterilize efficiently as much. Furthermore, the coupled core type optical fiber has an advantage that the fiber deterioration due to ultraviolet rays can be alleviated and the service life can be prolonged.

### (6) Solid Core Type Multi-Core Optical Fiber

In this optical fiber, a plurality of solid cores 52 with a high refractive index are disposed in the clad 60 apart from each other. This optical fiber guides light in a state where the influence of the light wave coupling can be ignored by sufficiently reducing the light wave coupling between the solid cores 52. Therefore, the solid core type multi-core optical fiber has the advantage that each core can be handled as an independent waveguide.

### (7) Hole Assist Type Multi-Core Optical Fiber

The optical fiber has a structure in which a plurality of hole structures and core regions of the above-mentioned (2) are disposed in the clad 60.

### (8) Hole Structure Type Multi-Core Optical Fiber

The optical fiber has a structure in which a plurality of hole structures of the above-mentioned (3) are disposed in the clad 60.

### (9) Hollow Core Type Multi-Core Optical Fiber

The optical fiber has a structure in which a plurality of hole structures of the above-mentioned (4) are disposed in the clad 60.

### (10) Coupled Core Type Multi-Core Optical Fiber

The optical fiber has a structure in which a plurality of hole structures of the above-mentioned (5) are disposed in the clad 60.

### Embodiment 2

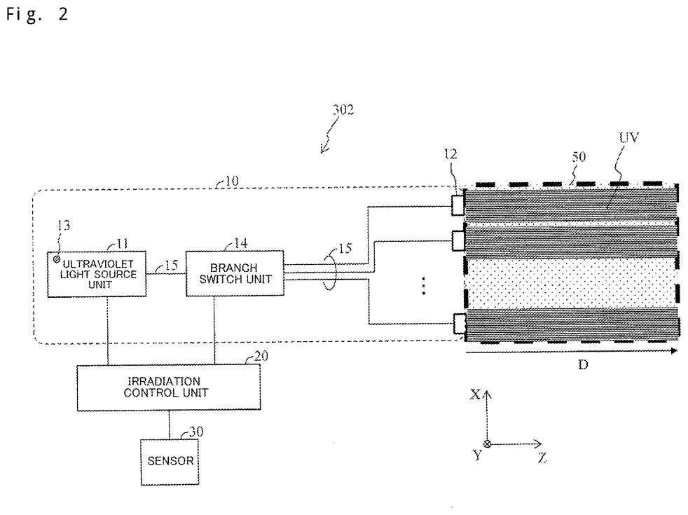

FIG. 2 is a diagram illustrating an ultraviolet light irradiation system 302 according to the present embodiment. The ultraviolet light irradiation system 302 is an ultraviolet light irradiation system including an ultraviolet light irradiation unit 10 for guiding the ultraviolet light UV in a collimated state into a desired space 50, in which the ultraviolet light irradiation unit 10 includes

one ultraviolet light source unit 11,
a plurality of condensing components 12 arranged on a straight line or a plane at arbitrary intervals and emitting the supplied ultraviolet light as ultraviolet light in the col limited state, and
a branch switch unit 14 that branches the ultraviolet light output from the ultraviolet light source unit 11 and supplies the branched ultraviolet light to the respective condensing components 12, or supplies the ultraviolet light output from the ultraviolet light source unit 11 to the respective condensing components 12 in order.

The ultraviolet light irradiation system 302 differs from the ultraviolet light irradiation system 301 of Embodiment 1 in that the ultraviolet light source unit 11 is one and the branch switch unit 14 is provided.

The ultraviolet light source unit 11 and the branch switch unit 14, and the branch switch unit 14 and each condensing component 12 are connected by an optical fiber 15. The optical fiber 15 is an optical fiber capable of guiding ultraviolet light. For example, in the optical fiber 15, the core is made of pure quartz glass having a high OH group concentration, and the clad is made of quartz glass having a refractive index lower than that of the core. In a clad region, the refractive index is effectively reduced by glass whose refractive index is reduced by fluorine or the like or a plurality of holes. In addition, the optical fiber 15 may have a hollow core structure. In this structure, the clad has a photonic band gap structure or an anti-resonant structure in which a wavelength band used is a transmission region.

Specifically, as the optical fiber 15, an optical fiber having a cross section as illustrated in FIG. 4 can be used. In addition to the solid type optical fiber using a general additive such as that illustrated in (1) of FIG. 4, optical fibers having a hole structure described in (2) to (4) of FIG. 4, multi-core optical fibers having a plurality of core regions described in (5) and (6) of FIG. 4, or optical fibers having a structure obtained by combining them ((7) to (10) of FIG. 4) may be used.

The branch switch unit 14 branches the power of the ultraviolet light from the ultraviolet light source unit 11 at approximately the same ratio and supplies the branched light to the respective condensing components 12. Alternatively, the branch switch unit 14 is an optical switch, and the ultraviolet light from the ultraviolet light source unit 11 may be supplied to the condensing component 12 in order at a constant time interval. In this case, an irradiation control 20 changes a switching destination of the branch switch unit 14. The fixed time interval is preferably an interval in which the ultraviolet light can be supplied to all the condensing components 12 within 0.1 seconds. For example, if there are eight condensing components 12, the branch switch unit 14 switches the condensing component 12, which is the supply destination of the ultraviolet light, every time shorter than 12.5 ms.

As described in Embodiment 1, by arranging the condensing components 12 in a row in the X-direction, the curtain-like space 50 of ultraviolet light can be formed. Further, when the condensing components 12 are also arranged in the Y-direction (the condensing components 12 can be arranged on a plane), the width in the Y-direction of the space 50 can be widened to the width equivalent to several of the arranged ultraviolet light UV. That is, the thickness of the curtain of the ultraviolet light can be increased.

The condensing component 12 is a collimator lens for collimating light emitted from the optical fiber 15. The collimator lens is installed at the emission end of the optical fiber 15. Another configuration of the condensing component 12 may be a lens in which the outgoing end of the optical fiber 15 is processed into a spherical surface or a lens in which the refractive index distribution of the outgoing end of the optical fiber 15 is processed into a graded shape. In the latter two cases, it is not necessary to consider the coupling efficiency between the optical fiber 15 and the lens and the characteristic deterioration due to ultraviolet light, sot hat it is possible to achieve low loss and high reliability, which is preferable.

As described in Embodiment 1, the space 50 is irradiated with the ultraviolet light UV, so that decontamination can be performed. That is, the ultraviolet light irradiation system 302 is disposed at an arbitrary place to form the space 50 which is the curtain of the ultraviolet light, and the human body or the object can be decontaminated simply by passing through the space 50. In addition, the ultraviolet light irradiation system 302 is disposed in a room larger than the space 50, and the room can be divided with respect to bacteria and viruses in the space 50.

As compared with the ultraviolet light irradiation system 301 of Embodiment 1, the ultraviolet light irradiation system 302 can suppress the number of light sources, the cost, and deterioration in reliability due to maintenance and failure of the light sources.

### Embodiment 3

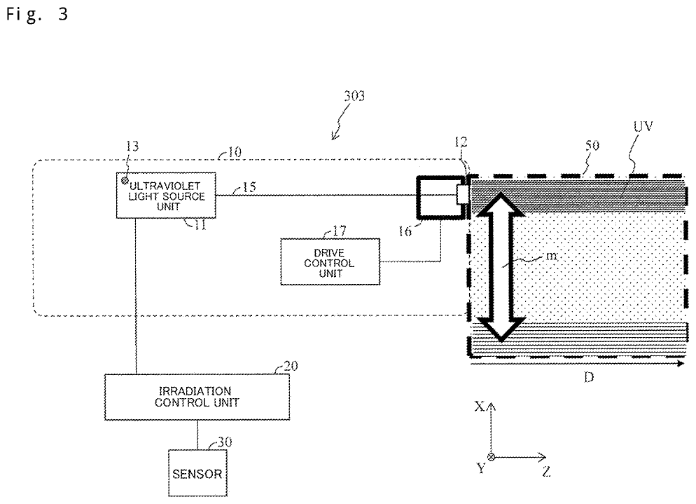

FIG. 3 is a diagram illustrating an ultraviolet light irradiation system 303 according to the present embodiment. The ultraviolet light irradiation system 303 is an ultraviolet light irradiation system including an ultraviolet light irradiation unit 10 for guiding the ultraviolet light UV in a collimated state into a desired space 50, in which the ultraviolet light irradiation unit 10 includes

one ultraviolet light source unit 11,
one condensing component 12 that emits the ultraviolet light output from the ultraviolet light source unit 11 as ultraviolet light in the collimated state, and
a drive control unit 17 that causes the condensing component 12 to scan on a straight line or a plane.

The ultraviolet light irradiation system 303 differs from the ultraviolet light irradiation system 301 of Embodiment 1 in that the ultraviolet light source unit 11 is one and one light condensing component 12 is scanned.

The ultraviolet light source unit 11 and the condensing component 12 are connected by the optical fiber 15. The optical fiber 15 is held on the condensing component 12 side by a holding part 16. Further, the drive control unit 17 can move the holding part 16 to an arbitrary position. For example, by moving the holding part 16 in the X-direction on a straight line, the ultraviolet light UV can be moved in a movable region m, and the space 50 can be formed by a depth D and the movable region m. In addition, when the holding part 16 is also moved in the Y-direction on the plane, the width in the Y-direction of the space 50 can be widened.

Specifically, as the optical fiber 15, an optical fiber having a cross section as illustrated in FIG. 4 can be used. In addition to the solid type optical fiber using a general additive such as that illustrated in (1) of FIG. 4, optical fibers having a hole structure described in (2) to (4) of FIG. 4, multi-core optical fibers having a plurality of core regions described in (5) and (6) of FIG. 4, or optical fibers having a structure obtained by combining them ((7) to (10) of FIG. 4) may be used.

A movement time of the holding part 16 from the movement start position to the movement end position is preferably 0.1 seconds or shorter. For example, in a case where the holding part 16 is moved by 10 cm in the X-direction, it is preferable to move the holding part 16 at a speed of 1 m/s or longer.

As described in Embodiment 1, the space 50 is irradiated with the ultraviolet light UV, so that decontamination can be performed. That is, the ultraviolet light irradiation system 303 is disposed at an arbitrary place to form the space 50, and the human body or the object can be decontaminated simply by passing through the space 50. In addition, the ultraviolet light irradiation system 303 is disposed in a room larger than the space 50, and the room can be divided with respect to bacteria and viruses in the space 50.

With respect to the ultraviolet light irradiation system 301 of Embodiment 1 or the ultraviolet light irradiation system 302 of Embodiment 2, the ultraviolet light irradiation system 303 can reduce the number of ultraviolet light sources, the number of optical fibers, and the number of condensing components, can reduce the cost, and can suppress the deterioration of reliability due to the maintenance and failure of the light source.

In the present embodiment, it has been described that the emitting direction of the ultraviolet light UV is fixed in the Z-direction and the condensing component 12 is moved in the X-direction or in the XY plane. The present invention is not limited to this aspect, and for example, the holding part 16 may be a swing mechanism to change the emission direction of the ultraviolet light UV at any time. In a case of the aspect, the movable part can be simplified and the direction can be controlled at a high speed, which is preferable.

### Example

A specific example will be described below.

A first specific example is an example in which the ultraviolet light irradiation system 302 is disposed between seats such as bleacher seats in a movie theater. The ultraviolet light source unit 11 is arranged in a place other than a viewing area, propagates the ultraviolet light through the optical fiber 15, and distributes the ultraviolet light to a plurality of ultraviolet light irradiation systems 302 by a branching device 24. Further, the ultraviolet light irradiation system 302 also branches the ultraviolet light by the branch switch unit 14 and emits the ultraviolet light from the condensing component 12. As a result, the space 50, a curtain of ultraviolet light is generated between the seats, and infection by adjacent persons can be prevented.

A second example is an example in which the ultraviolet light irradiation system 303 is disposed at an entrance of a closed space such as a store or transportation. The upper part of the entrance is scanned by the holding part 16. The space 50 is formed at the entrance of the store by the ultraviolet light UV, and a person entering the store only passes the entrance to complete decontamination.

### REFERENCE SIGNS LIST

10 Ultraviolet light irradiation unit
11 Ultraviolet light source unit
12, 2 Condensing component
13 Display unit
14 Branch switch unit
15 Optical fiber
16 Holding part
17 Drive control unit
20 Irradiation control unit
24 Branching device
30 Sensor
50 Decontamination space
52 Solid core
52a Region
53 Hole
53a Hole group
60 Clad
301 to 303 Ultraviolet light irradiation system

1. An ultraviolet light irradiation system comprising:
ultraviolet light irradiation units that guide ultraviolet light in a collimated state into a desired space, wherein
the ultraviolet light irradiation units are arranged on a straight line or a plane at arbitrary intervals, and
each of the ultraviolet light irradiation units includes an ultraviolet light source unit that emits the ultraviolet light and a condensing component that causes the ultraviolet light incident from the ultraviolet light source unit directly or via an optical fiber to become ultraviolet light in the collimated state.

2. An ultraviolet light irradiation system comprising:
ultraviolet light irradiation units that guide ultraviolet light in a collimated state into a desired space, wherein
the ultraviolet light irradiation unit includes
one ultraviolet light source unit,
a plurality of condensing components arranged on a straight line or a plane at arbitrary intervals and emitting the supplied ultraviolet light as ultraviolet light in the collimated state,
a branch switch unit that branches the ultraviolet light output from the ultraviolet light source unit and supplies the branched ultraviolet light to the respective condensing components, or supplies the ultraviolet light output from the ultraviolet light source unit to the respective condensing components in order, and
an optical fiber for supplying the ultraviolet light output from the ultraviolet light source unit to the condensing component.

3. An ultraviolet light irradiation system comprising:
ultraviolet light irradiation units that guide ultraviolet light in a collimated state into a desired space, wherein
the ultraviolet light irradiation unit includes
one ultraviolet light source unit,
one condensing component that emits the ultraviolet light output from the ultraviolet light source unit as ultraviolet light in the collimated state,
an optical fiber for supplying the ultraviolet light output from the ultraviolet light source unit to the condensing component, and
a drive control unit that causes the condensing component to scan on a straight line or a plane.

4. The ultraviolet light irradiation system according to claim 1, further comprising:
a sensor for sensing an object to be irradiated in the desired space; and
an irradiation control unit that controls output or non-output of ultraviolet light from the ultraviolet light source unit based on a signal of the sensor.

5. The ultraviolet light irradiation system according to claim 1, further comprising:
a display unit configured to display an output state of the ultraviolet light of the ultraviolet light source unit.

6. The ultraviolet light irradiation system according to claim 1, wherein
the optical fiber includes any one of a solid core optical fiber, a hole assist optical fiber, a hole structure optical fiber, a hollow core optical fiber, a coupled core type optical fiber, a solid core type multi-core optical fiber, a hole assist type multi-core optical fiber, a hole structure type multi-core optical fiber, a hollow core type multi-core optical fiber, and a coupled core type multi-core optical fiber.

7. The ultraviolet light irradiation system according to claim 1, wherein
the ultraviolet light in the collimated state is collimated by a collimator lens, and
the collimator lens is an optical fiber whose tip is processed into a spherical shape or an optical fiber having a graded refractive index distribution at the tip.
